(19) United States
(12) Patent Application Publication
(10) Pub. No.: US 2026/0234739 A1
(43) Pub. Date: Aug. 13, 2026

(54) HCV DETECTION

(71) Applicant: DIAGNOSTICS FOR THE REAL WORLD, LTD, San Jose, CA (US)

(72) Inventors: Sonny Michael ASSENNATO, Little Chesterford (GB); Allyson Victoria RITCHIE, Little Chesterford (GB)

(73) Assignee: DIAGNOSTICS FOR THE REAL WORLD, LTD, San Jose, CA (US)

(21) Appl. No.: 19/563,714

(22) Filed: Mar. 11, 2026

Related U.S. Application Data
(63) Continuation of application No. 17/299,107, filed on Dec. 3, 2019, now abandoned.

(30) Foreign Application Priority Data
Dec. 3, 2018 (GB) 1819726.9

(51) Int. Cl. C12Q 1/70; C12Q 1/6806

(52) U.S. Cl. CPC C12Q 1/707; C12Q 1/6806; C12Q 2600/112

(57) ABSTRACT

Methods for detecting Hepatitis C virus (HCV) nucleic acid are described. The methods are useful for point-of-care (POC) testing, and provide rapid tests able to detect several different HCV genotypes. Kits, primers, probes, sets of primers, sets of oligonucleotides, and oligonucleotides, and their use in the methods, are also described.

### CROSS REFERENCE TO RELATED APPLICATIONS

This application is a continuation of U.S. patent application Ser. No. 17/299,107, filed Jun. 2, 2021, which is a National Stage Entry of International Application No. PCT/US2019/064182, filed Dec. 3, 2019, which claims priority from GB Application No. 1819726.9, filed Dec. 3, 2018, the entire contents of all of which are incorporated by reference herein.

This invention relates to methods for detecting Hepatitis C virus (HCV) nucleic acid, particularly for point-of-care (POC) testing, and to kits, primers, probes, sets of primers, sets of oligonucleotides, and oligonucleotides, and their use in the methods.

### INCORPORATION BY REFERENCE OF SEQUENCE LISTING

The Sequence Listing in XML format, named 39656Z_SequenceListing.xml of 69,632 bytes, created on Mar. 3, 2026, and submitted to the United States Patent and Trademark Office via Patent Center, is incorporated herein by reference.

Hepatitis C is an infectious disease caused by hepatitis C virus (HCV) that primarily affects the liver. During the initial infection there are often only mild or no symptoms. The virus persists in the liver in about 75% to 85% of those initially infected. Early on chronic infection typically has no symptoms. Over many years however, it often leads to liver disease and occasionally cirrhosis. In some cases, those with cirrhosis will develop complications such as liver failure, liver cancer, or dilated blood vessels in the oesophagus and stomach.

HCV is spread primarily by blood-to-blood contact associated with intravenous drug use, poorly sterilized medical equipment, needlestick injuries in healthcare, and transfusions. 143 million people (2%) worldwide were estimated to have been infected with hepatitis C as of 2015 (GBD 2015 Disease and Injury Incidence and Prevalence, Collaborators. (8 Oct. 2016). Lancet, 388 (10053): 1545-1602). It occurs most commonly in Africa and Central and East Asia. About 167,000 deaths due to liver cancer and 326,000 deaths due to cirrhosis occurred in 2015 due to hepatitis C (GBD 2015 Mortality and Causes of Death, Collaborators. (8 Oct. 2016). Lancet, 388 (10053): 1459-1544). There is no vaccine against hepatitis C. However, treatment of HCV infection has been revolutionized with the development and approval of potent and well-tolerated direct acting antiviral drug combinations. These therapies yield over 95% cure rates after 8 to 24 weeks of administration in most patient populations (Belperio, et al., 2017, Ann Intern Med 167, 499-5045; Falade-Nwulia, et al, 2017, Ann Intern Med 166, 637-648).

HCV is an enveloped, RNA virus of the family Flaviviridae. HCV particles comprise a lipid membrane envelope in which two viral envelope glycoproteins, E1 and E2, are embedded. They take part in viral attachment and entry into the cell. Within the envelope is an icosahedral core containing the RNA material of the virus. HCV has a positive sense single-stranded RNA genome, which consists of a single open reading frame that is 9,600 nucleotide bases long. This single open reading frame is translated to produce a single protein product of about 3,000 amino acids, which is then further processed by cellular and viral proteases into 10 smaller proteins that allow viral replication within the host cell, or assemble into the mature viral particles. At the 5′ and 3′ ends of the RNA are non-translated regions (NTRs), which are not translated into proteins, but are important to translation and replication of the viral RNA.

Structural proteins made by the hepatitis C virus include Core protein, E1 and E2. Nonstructural (NS) proteins include NS2, NS3, NS4A, NS4B, NS5A, and NS5B. The proteins are arranged along the genome in the following order: N terminal-core-envelope (E1)-E2-p7-NS2-NS3-NS4A-NS4B-NS5A-NS5B-C terminal. The core protein has 191 amino acids.

HCV has been classified into seven major genotypes (1-7) and 67 subtypes (Smith et al., 2014 (Hepatology 2014; 59; 318-327). Genotypes differ by 30-35% of the nucleotide sites over the complete genome (Ohno et al. (2007), J Clin Microbiol. 35 (1): 201-7). The difference in genomic composition of subtypes of a genotype is usually 20-25%. Subtypes 1a and 1b are found worldwide and cause 60% of all cases.

Diagnosis of HCV is by blood testing to look for antibodies to the virus or its RNA. Several methods of HCV detection have been approved by the US FDA:
Hepatitis C Virus Encoded Antigen (Anti-HCV Assay): ABBOTT HCV EIA 2.0 (Abbott Laboratories); Chiron RIBA HCV 3.0 Strip Immunoblot Assay (Chiron Corp); ABBOTT PRISM HCV (Abbott Laboratories);
Nucleic Acid Testing: UltraQual HCV RT-PCR Assay (National Genetics Institute); COBAS AmpliScreen HCV Test (Roche Molecular Systems, Inc); Procleix HIV-1/HCV Assay (Gen-Probe, Inc); Procleix Ultrio Assay (Gen-Probe, Inc); Procleix Ultrio Plus Assays (Gen-Probe, Inc); Hepatitis C Virus (HCV) Reverse Transcription (RT) Polymerase Chain Reaction (PCR) Assay (BioLife Plasma Services, L.P.);
ELISA Test: Ortho HCV Version 3.0 ELISA Test System (Ortho-Clinical Diagnostics, Inc).

These methods are not suitable for use in hard-to-reach populations and low resource settings, where HCV infection is prevalent, as they are laboratory-based, time-consuming and costly. Also, antibody-based tests can only detect HCV infection generally 6-12 weeks post infection. There is a need, therefore, for rapid tests that are suitable for use in hard-to-reach populations and low resource settings, and that can detect HCV as soon as possible after infection.

The Applicant has appreciated that rapid POC nucleic acid tests able to detect several different HCV genotypes can be provided by using nucleic acid amplification primers that hybridise specifically to conserved regions of the HCV core nucleic acid sequence (or the complement thereof).

According to the invention there is provided a method for determining whether a sample includes HCV nucleic acid, which comprises amplifying nucleic acid of the sample, or amplifying nucleic acid derived from nucleic acid of the sample, by an isothermal amplification reaction using a forward nucleic acid amplification primer and a reverse nucleic acid amplification primer, wherein each nucleic acid amplification primer hybridises specifically to HCV core nucleic acid sequence, or the complement thereof, that is conserved between at least HCV genotypes 1-6.

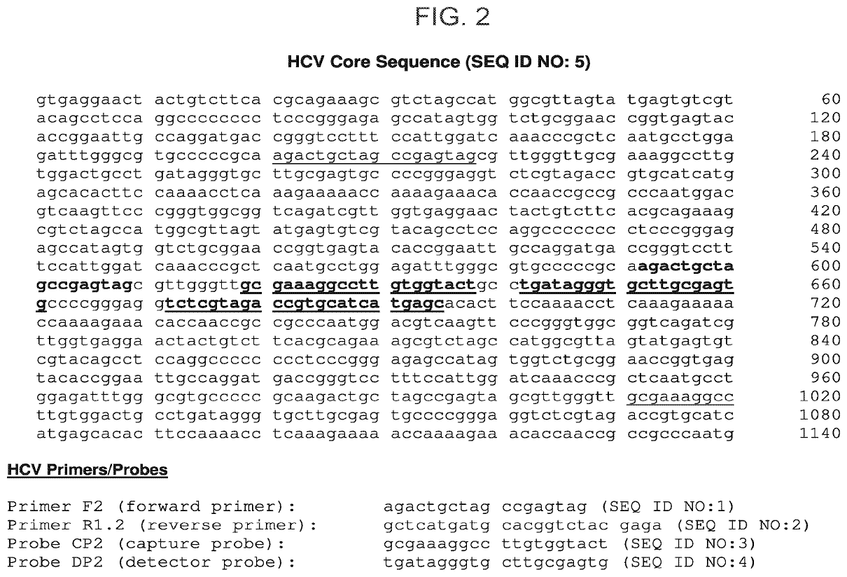

### HCV Core Nucleic Acid Sequence is Shown in FIG. 2.

Conserved sequences may be identified by homology search, using tools such as BLAST, HMMER and Infernal. Homology search tools may take an individual nucleic acid sequence as input, or use statistical models generated from multiple sequence alignments of known related sequences. Statistical models such as profile-HMMs, and RNA covariance models which also incorporate structural information, can be helpful when searching for more distantly related sequences. Input sequences are then aligned against a database of sequences from related individuals or other species. The resulting alignments are then scored based on the number of matching bases, and the number of gaps or deletions generated by the alignment. Acceptable conservative substitutions may be identified using substitution matrices such as PAM and BLOSUM. Highly scoring alignments are assumed to be from homologous sequences. The conservation of a sequence may then be inferred by detection of highly similar homologs over a broad phylogenetic range.

Optionally conserved HCV core nucleic acid sequence between different HCV genotypes is nucleic acid sequence that includes up to 2 mismatches per 20 nucleotides for each HCV genotype 1-6.

Optionally conserved HCV core nucleic acid sequence between different HCV genotypes is nucleic acid sequence that is identical for each HCV genotype 1-6.

Multiple sequence alignments can be used to visualise conserved sequences. The CLUSTAL format includes a plain-text key to annotate conserved columns of the alignment, denoting conserved sequence (*), conservative mutations (:), semi-conservative mutations (.), and non-conservative mutations ( ). Software such as MacVector can be used to perform multiple sequence alignments.

Optionally a nucleic acid amplification primer hybridises specifically to HCV core nucleic acid sequence if it hybridises under stringent conditions to HCV core nucleic acid sequence, or the complement thereof, that is conserved between at least HCV genotypes 1-6.

The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. The Tm is the temperature under defined ionic strength and pH, at which 50% of the target sequence hybridises to a perfectly matched primer or probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:
1) DNA-DNA hybrids (Meinkoth and Wahl, Anal. Biochem., 138: 267-284, 1984):

$${T}_{m} = 81.5 ° C . + 16.6 \times {{log}_{10} [ {Na}^{+} ]}^{a} + 0.41 \times \% [ G / {C}^{b} ] - 500 \times {[ {L}^{c} ]}^{- 1} - 0.61 \times \% formamide ;$$

2) DNA-RNA or RNA-RNA hybrids:

$${T}_{m} = 79.8 ° C . + 18.5 ( {{log}_{10} [ {Na}^{+} ]}^{a} ) + 0.58 ( \% G / {C}^{b} ) + 11.8 {( \% G / {C}^{b} )}^{2} - 820 / {L}^{c} ;$$

3) oligo-DNA or oligo-RNAs hybrids:
For <20 nucleotides: Tₘ=2(lₙ);
For 20-35 nucleotides: Tₘ=22+1.46(lₙ);
^{a} or for other monovalent cation, but only accurate in the 0.01-0.4 M range.
^{b} only accurate for % GC in the 30% to 75% range.
^{c} L=length of duplex in base pairs.
^{d} oligo, oligonucleotide; 1_{n,}=effective length of primer=2×(no. of G/C)+(no. of NT).

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical stringent conditions (also referred to as high stringency hybridisation conditions) for DNA hybrids longer than 50 nucleotides encompass hybridisation at 65° C. in 1×SSC or at 42° C. in 1×SSC and 50% formamide, followed by washing at 65° C. in 0.3×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

Optionally the forward nucleic acid primer comprises a nucleic acid sequence of: AGACTGCTAGCCGAGTAG (SEQ ID NO:1), or a nucleic acid sequence that has at least 90%, 91%, 92%, 93%, 94%, 95%, 96%, 97%, 98%, or 99% identity along its entire length with a nucleic acid sequence of SEQ ID NO:1.

Optionally the reverse nucleic acid primer comprises a nucleic acid sequence of: GCTCATGATGCACGGTCTACGAGA (SEQ ID NO:2), or a nucleic acid sequence that has at least 90%, 91%, 92%, 93%, 94%, 95%, 96%, 97%, 98%, or 99% identity along its entire length with a nucleic acid sequence of SEQ ID NO:2.

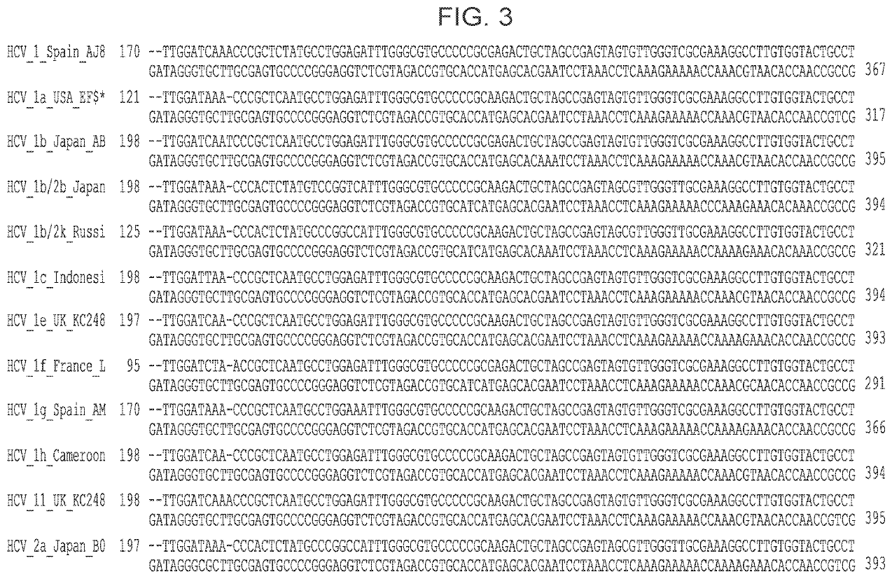

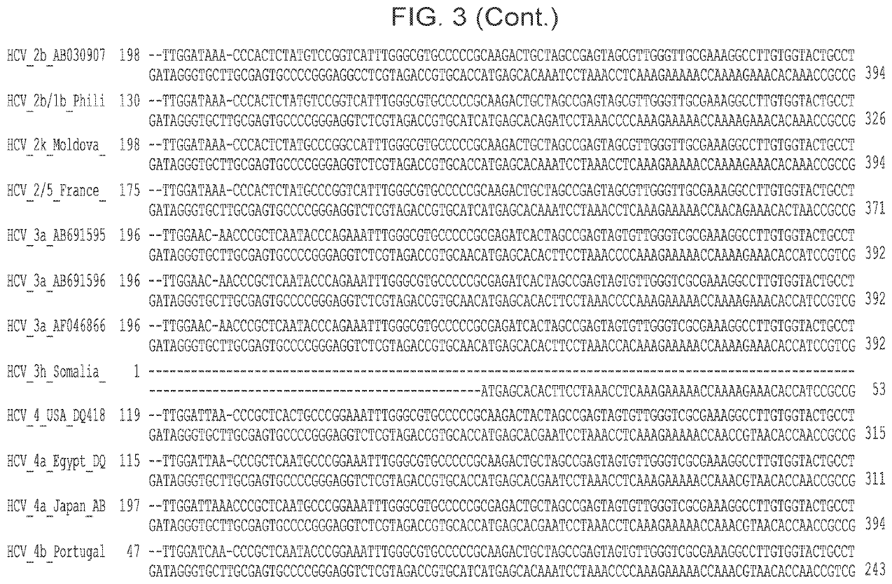

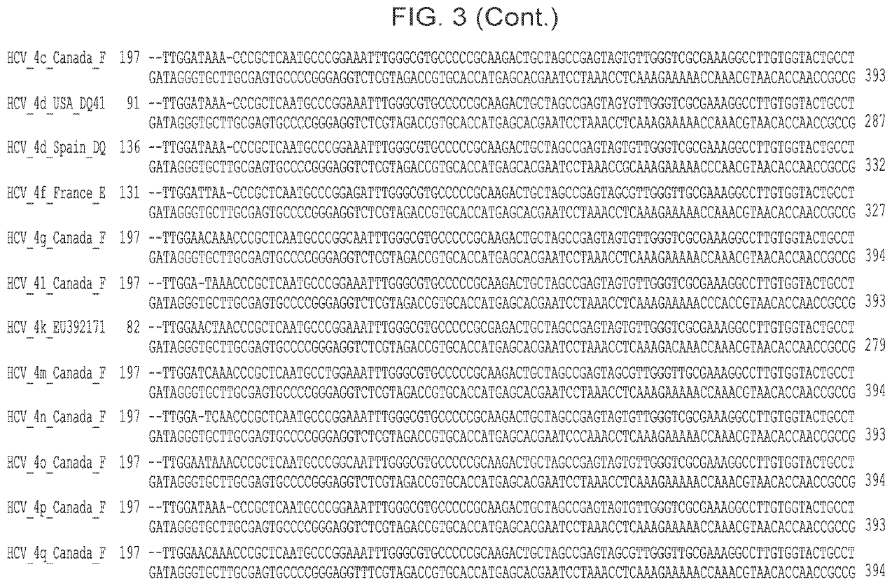

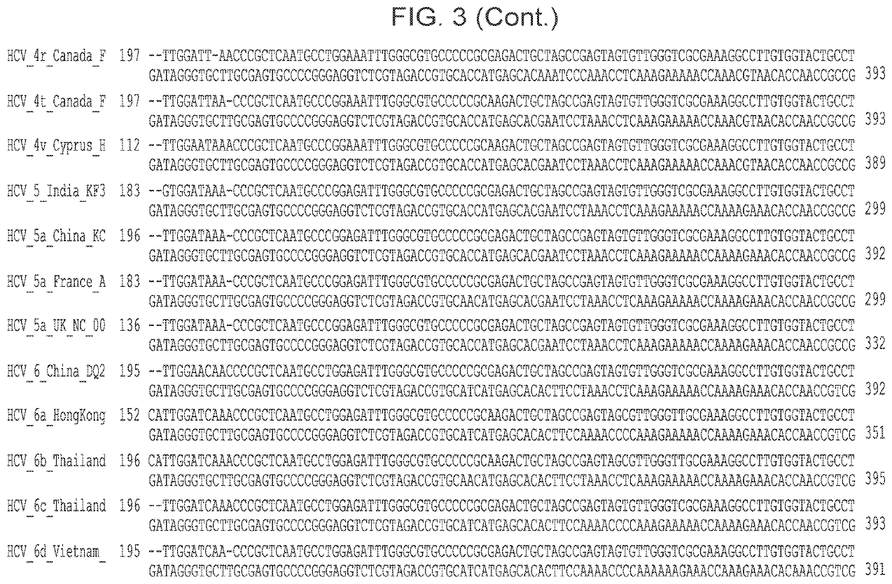

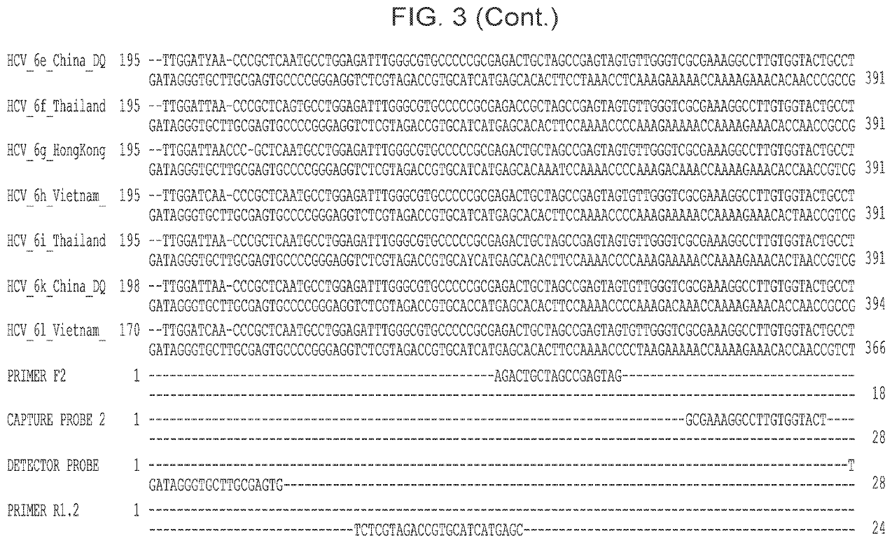

The location in the HCV core region of sequence corresponding to the sequence of SEQ ID NO:1 (Primer F2, forward primer), and sequence corresponding to the reverse complement of sequence of SEQ ID NO:2 (Primer R1.2, reverse primer), is shown in FIG. 2. A sequence alignment of HCV core nucleic acid sequence for HCV genotypes 1-6 is shown in FIG. 3, as well as the positioning of sequence corresponding to SEQ ID NO:1 (Primer F2) and of sequence corresponding to the reverse complement of SEQ ID NO:2 (Primer R1.2).

Nucleic acid may be derived from nucleic acid of the sample, for example by reverse transcribing HCV core nucleic acid of the sample, and amplifying a product of the reverse transcription by an isothermal nucleic acid amplification reaction using the forward and reverse nucleic acid amplification primers.

Optionally a method of the invention further comprises reverse transcribing HCV RNA of the sample, and amplifying a product of the reverse transcription by an isothermal amplification reaction using the forward and reverse nucleic acid amplification primers.

Any suitable method of isothermal nucleic acid amplification may be used in methods of the invention. Several suitable methods of isothermal nucleic acid amplification are known to the skilled person. Optionally the isothermal nucleic acid amplification is a transcription-based amplification. Such methods involve amplification of an RNA template using reverse transcriptase (RT), RNase H, and RNA polymerase activities, and include nucleic acid sequence-based amplification (NASBA), transcription-mediated amplification (TMA), and self-sustained sequence replication (3SR) (Chan and Fox, Rev. Med. Microbiol. 10: 185-196 (1999); Guatelli et al., Proc. Natl. Acad. Sci. 87: 1874-1878 (1990); Compton, Nature 350:91-92 (1991)). NASBA and 3SR use RT from Avian Myeloblastosis Virus (AMV) (which also has RNaseH activity), RNase H from E. coli, and T7 RNA polymerase. TMA uses Moloney Murine Leukemia Virus (MMLV) RT (which also has RNase H activity), and T7 RNA polymerase.

Isothermal amplification methods, such as transcription-based amplification methods, have several advantages over amplification using a Polymerase Chain Reaction (PCR). The reactions occur simultaneously in a single tube, and are carried out under isothermal conditions so a thermocycler is not required. The amplification reaction is faster than PCR (1×10⁹-fold amplification can be seen after five cycles, compared with 1×10⁶-fold amplification after 20 cycles for PCR). DNA background does not interfere with transcription-based amplification, and so these methods are not affected by double stranded DNA contamination. The amplification product is single stranded and can be detected without any requirement for strand separation.

Optionally the reverse nucleic acid primer further comprises a promoter sequence for a DNA-dependent RNA polymerase at its 5′-end. Such a primer can be used for the reverse transcription and for a transcription-based isothermal amplification reaction, thereby minimising the number of primers required to carry out reverse transcription and isothermal nucleic acid amplification.

For example, the promoter sequence may be a T7 promoter sequence: 5′ TAATACGACTCACTATAG 3′ (SEQ ID NO:6). T7 RNA polymerase starts transcription at the underlined G in the promoter sequence. The polymerase then transcribes using the opposite strand as a template from 5′->3′. The first base in the transcript will be a G.

For example, the reverse nucleic acid primer with a T7 promoter sequence at its 5′-end may comprise a nucleic acid sequence of: GCTCATGATGCACGGTCTACGAGA TAATACGACTCACTATAG (SEQ ID NO:7).

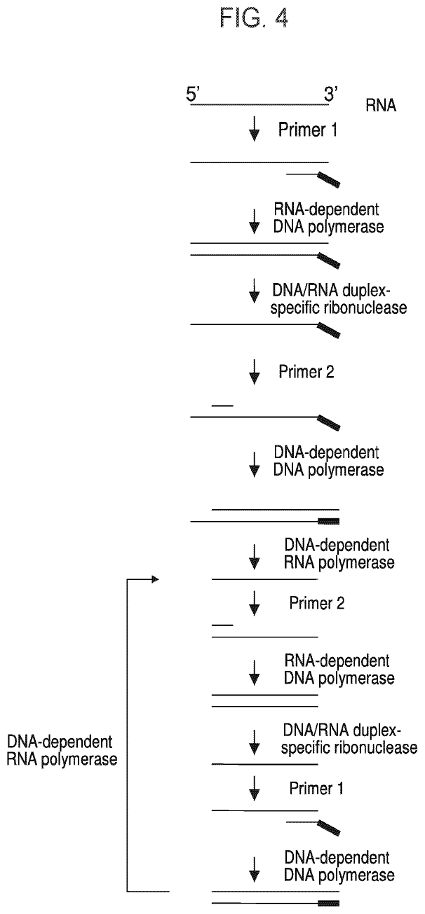

A transcription-based isothermal amplification reaction suitable for use in methods of the invention is described below, with reference to FIG. 4.

An antisense Primer 1 comprises nucleic acid sequence complementary to a portion of a target RNA so that the primer can hybridise specifically to the target RNA (for example, SEQ ID NO:2, Primer R1.2, reverse primer), and a single stranded-version of a promoter sequence for a DNA-dependent RNA polymerase at its 5′-end (for example, SEQ ID NO:7, T7 promoter sequence). Primer 1 is annealed to the RNA target. An RNA-dependent DNA polymerase extends Primer 1 to synthesise a complementary DNA (cDNA) copy of the RNA target. A DNA/RNA duplex-specific ribonuclease digests the RNA of the RNA-cDNA hybrid. A sense Primer 2 comprises nucleic acid sequence complementary to a portion of the cDNA. Primer 2 is annealed to the cDNA downstream of the part of the cDNA formed by Primer 1. Primer 2 is extended by a DNA-dependent DNA polymerase to produce a second DNA strand which extends through the DNA-dependent RNA polymerase promoter sequence at one end (thereby forming a double stranded promoter). This promoter is used by a DNA-dependent RNA polymerase to synthesise a large number of RNAs complementary to the original target sequence. These RNA products then function as templates for a cyclic phase of the reaction, but with the primer annealing steps reversed, i.e., Primer 2 followed by Primer 1.

In a variation of this method, Primer 2 may also include a single stranded version of a promoter sequence for the DNA-dependent RNA polymerase. This results in production of RNAs with the same sense as the original target sequence (as well as RNAs complementary to the original target sequence).

In some conventional isothermal transcription-based amplification reactions it is known to cleave the target RNA at the 5′-end before it serves as the template for cDNA synthesis. An enzyme with RNase H activity is used to cleave the RNA portion of an RNA-DNA hybrid formed by adding an oligonucleotide (a cleavage oligonucleotide) having a sequence complementary to the region overlapping and adjacent to the 5′-end of the target RNA. The cleavage oligonucleotide may have its 3′-terminal-OH appropriately modified to prevent extension reaction. Whilst in some embodiments of the invention a cleavage oligonucleotide could be used, it is preferred that a method of the invention is carried out in the absence of a cleavage oligonucleotide thereby simplifying the amplification reaction and the components required.

Isothermal nucleic acid amplification is advantageous because it can readily be used in resource-limited settings. Such methods do not require the use of thermal cyclers which may not be available in resource-limited settings. Examples of suitable methods are described in WO 2008/090340 and Lee et al., Journal of Infectious Diseases 2010; 201(S1):S65-S71.

Examples of suitable reagents for carrying out reverse transcription of RNA, and for isothermal amplification of a product of the reverse transcription, are given in WO 2008/090340, and include, for example, the following enzyme activities: an RNA-dependent DNA polymerase, a DNA-dependent DNA polymerase, a DNA/RNA duplex-specific ribonuclease, and a DNA-dependent RNA polymerase.

It will be appreciated that in addition to the required enzyme activities, it will also be necessary to provide appropriate nucleotide triphosphates (for transcription-based amplifications ribonucleotide triphosphates (rNTPs, i.e. rATP, rGTP, rCTP, and rUTP), and deoxyribonucleotide triphosphates (dNTPs, i.e. dATP, dGTP, dCTP, and dTTP) are required), appropriate primers for specific amplification of the target nucleic acid, a suitable buffer for carrying out the amplification reaction, and any necessary cofactors (for example magnesium ions) required by the enzyme activities. Examples of suitable buffers include Tris-HCl, HEPES, or acetate buffer. A suitable salt may be provided, such as potassium chloride or sodium chloride. Suitable concentrations of these components may readily be determined by the skilled person. Suitable rNTP concentrations are typically in the range 0.25-5 mM, or 0.5-2.5 mM. Suitable dNTP concentrations are typically in the range 0.25-5 mM dNTP, or 0.5-2.5 mM. Suitable magnesium ion concentrations are typically in the range 5-15 mM.

Some conventional transcription-based amplification methods use very high amounts of T7 RNA polymerase (for example 142 or more units, where one unit incorporates 1 nmole of labelled nucleotide into acid insoluble material in 1 hour at 37° C. under standard assay conditions, such as: 40 mM Tris-HCl (pH8.0), 50 mM NaCl, 8 mM MgCl₂, 5 mM DTT, 400 μM rNTPs, 400 μM [³H]-UTP (30 cpm/pmoles), 20 μg/ml T7 DNA, 50 μg/ml BSA, 100 μl reaction volume, 37° C., 10 min.). Methods of the invention can be carried out using significantly less T7 RNA polymerase than such conventional methods, thereby reducing cost. For example, methods of the invention can be carried out using less than 142 units of a DNA-dependent RNA polymerase (for example T7 RNA polymerase), suitably less than 100 units or less than 50 units, such as 30-40 units.

Optionally nucleic acid of the sample is isolated before reverse transcribing HCV RNA of the sample present in the isolated nucleic acid.

Many suitable methods for isolation of nucleic acid are known to the skilled person. Some methods use chaotropic agents, such as guanidinium thiocyanate, and organic solvents to lyse cells, and denature proteins. For example, Boom et al. (Journal of Clinical Microbiology, 1990, Vol. 28(3): 495-503) describes methods in which a sample is contacted with silica particles in the presence of a lysis/binding buffer containing guanidinium thiocyanate. Released nucleic acid binds to the silica particles, which are then washed with a wash buffer containing guanidinium thiocyanate, then with ethanol, and then acetone. The bound nucleic acid is subsequently eluted in an aqueous low salt buffer (Tris-HCl, EDTA, pH 8.0).

Some methods avoid the requirement for chaotropic salts and organic solvents. For example, Hourfar et al. (Clinical Chemistry, 2005, 51(7): 1217-1222) describes methods in which a sample is mixed with magnetic silica particles in the presence of a lysis/binding buffer containing a kosmotropic salt (ammonium sulphate) before addition of proteinase K. Following separation, the magnetic particles are washed with wash buffer containing proteinase K, and eluted in elution buffer (Tris-HCl, pH 8.5) at 80° C. Other suitable methods are described in WO 2010/015835.

Isolation of nucleic acid may be carried out using conventional binding buffers and/or elution buffers for use with a solid phase that is able to bind the nucleic acid in the presence of binding buffer at a first pH, and from which the nucleic acid can be eluted at a second pH.

Optionally the solid phase comprises an ionisable group, which changes charge according to the ambient conditions. The pKa of the ionisable group is appropriate to the conditions at which it is desired to bind nucleic acid to and release nucleic acid from the solid phase. Generally, nucleic acid will bind to the solid phase at a pH below or roughly equal to the pKa, and will be released at a higher pH (usually above the pKa). Suitable solid phases for binding a nucleic acid at a first pH, and elution of bound nucleic acid at a second pH that is higher than the first pH, are well known to those of ordinary skill in the art. For example, at the first pH the solid phase may comprise a positive charge, and at the second pH the solid phase may have a less positive, neutral, or negative charge. Alternatively or additionally, at the first pH the solid phase may comprise a neutral or less negative charge, and at the second pH the solid phase may have a negative or more negative charge. Such changes in charge allow the nucleic acid to be adsorbed to the solid phase at the first pH, and released at the second pH.

For example, the solid phase may comprise a negatively ionisable group with a pKa between the first and second pH. Nucleic acid will bind to the solid phase when the solid phase is neutral or less negatively charged, and will be released when the solid phase is negatively or more negatively charged. Alternatively, or additionally, the solid phase may comprise a positively ionisable group with a pKa between the first and second pH. Nucleic acid will bind to the solid phase when the solid phase is positively charged, and will be released when the solid phase is neutral or less positively charged.

Examples of solid phases that may be used for extraction of nucleic acid include solid phases that comprise inorganic oxides, such as silica or glass (for example, as described in Boom et al, or Hourfar et al), or aluminium oxide, sugar polymers, or charge-switch materials (for example, as described in WO 02/48164).

The solid phase may be in any suitable form, for example comprising a membrane, gel, or particles, for example magnetic particles. Silica membrane or gel, and magnetic silica particles are preferred examples. Silica membrane is particularly preferred. This is less expensive than magnetic silica particles (used for example by Hourfar, et al.) and does not require refrigerated storage, unlike magnetic silica particles.

The solid phase may be a solid phase to which binding of nucleic acid is enhanced by the presence of a kosmotropic agent. Optionally binding of the nucleic acid to the solid phase is carried out in the presence of a kosmotropic agent. Such agents are known to enhance binding of nucleic acid to solid phases such as silica-based solid phases.

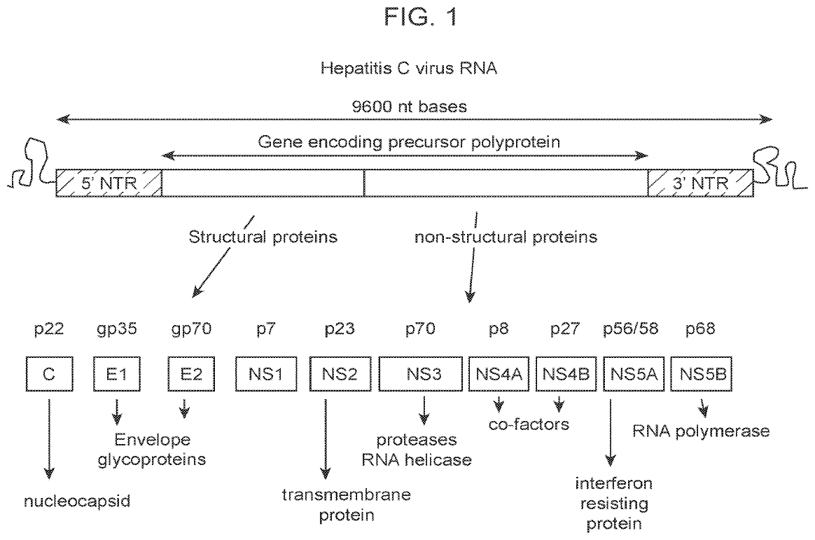

The terms “chaotropic” and “kosmotropic” agent originate from the Hofmeister series (Cacace et al., Q Rev Biophys 1997; 30:241-77), which divides these agents depending on their influence on the structure of macromolecules and water. A chaotrope may be defined as a substance that breaks solvent structure, and a kosmotrope as a substance that enhances solvent structure. FIG. 1 of Cacace et al shows the Hofmeister series and commonly occurring organic solutes with effects on protein structure/function. Examples of chaotropic agents are known to those in the art, and include sodium iodide, sodium perchlorate, guanidinium thiocyanate and guanidinium hydrochloride. Examples of kosmotropic agents are known to those in the art, and include ammonium sulphate and lithium chloride.

Optionally lysis is carried out using the binding buffer. Binding buffers that may be used for cell lysis are known to those of ordinary skill in the art. The lysis buffer used by Boom et al. comprises guanidinium thiocyanate, Tris hydrochloride, pH 6.4, EDTA (adjusted to pH 8), and Triton X-100. Optionally, the lysis buffer does not include a chaotropic agent. For example, a lysis/binding buffer that comprises a kosmotropic agent may be used. Optionally the buffer is an acidic buffer, suitably a strong acidic buffer with a pKa (25° C.) in the range 3-5.

Optionally a method of the invention further comprises capturing a product of the isothermal amplification reaction by hybridising nucleic acid of the product to a nucleic acid capture probe, wherein the capture probe hybridises specifically to HCV core nucleic acid sequence, or the complement thereof, that is conserved between at least HCV genotypes 1-6.

Optionally the capture probe hybridises specifically to HCV core nucleic acid sequence if it hybridises under stringent conditions to HCV core nucleic acid sequence, or the complement thereof, that is conserved between at least HCV genotypes 1-6.

Optionally the capture probe comprises a nucleic acid sequence of: GCGAAAGGCCTTGTGGTACT (SEQ ID NO:3), or a nucleic acid sequence that has at least 90%, 91%, 92%, 93%, 94%, 95%, 96%, 97%, 98%, or 99% identity along its entire length with a nucleic acid sequence of SEQ ID NO:3, or the complement thereof.

Optionally a method of the invention further comprises detecting a product of the isothermal amplification reaction by hybridising the product to a nucleic acid detector probe, wherein the detector probe hybridises specifically to HCV core nucleic acid sequence, or the complement thereof, that is conserved between at least HCV genotypes 1-6.

Optionally the detector probe hybridises specifically to HCV core nucleic acid sequence if it hybridises under stringent conditions to HCV core nucleic acid sequence, or the complement thereof, that is conserved between at least HCV genotypes 1-6.

Optionally the detector probe comprises a nucleic acid sequence of: TGATAGGGTGCTTGCGAGTG (SEQ ID NO:4), or a nucleic acid sequence that has at least 90%, 91%, 92%, 93%, 94%, 95%, 96%, 97%, 98%, or 99% identity along its entire length with a nucleic acid sequence of SEQ ID NO:4, or the complement thereof.

The location in the HCV core region of sequence corresponding to the sequence of SEQ ID NO:3 (Probe CP2, capture probe) and SEQ ID NO:4 (Probe DP2, detector probe), is shown in FIG. 2. A sequence alignment of HCV core nucleic acid sequence for HCV genotypes 1-6 is shown in FIG. 3, as well as the positioning of sequence corresponding to SEQ ID NO:3 (Capture Probe 2) and SEQ ID NO:4 (Detector Probe).

A capture probe, and a detector probe, hybridises specifically to HCV core nucleic acid sequence, for example, if it does not hybridise to other nucleic acid (i.e. non-HCV core nucleic acid, including HCV nucleic acid outside the core region) present in the isothermal amplification reaction under stringent hybridisation conditions.

Sequence identity between nucleic acid sequences can be determined by comparing an alignment of the sequences. When an equivalent position in the compared sequences is occupied by the same nucleotide, then the molecules are identical at that position. Scoring an alignment as a percentage of identity is a function of the number of identical nucleotides at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score.

Optionally the detector probe is labelled with a visually detectable label (i.e. a label that is visually detectable without the use of instrumentation). Examples of suitable visually detectable labels include colloidal metal sol particles, latex particles, or textile dye particles. An example of colloidal metal sol particles is colloidal gold particles.

A product of the isothermal nucleic acid amplification may be labelled with a visually detectable label, and captured and detected using a chromatographic test strip, for example as described in WO 2008/090340, and Lee et al., Journal of Infectious Diseases 2010; 201(S1):S65-S71.

Optionally the sample is a liquid sample. Optionally the sample is a biological sample, for example a liquid biological sample, obtained from a subject suspected of being infected with HCV. Optionally the sample is a blood or a plasma sample obtained from a subject suspected of being infected with HCV.

Optionally a method of the invention is an in vitro method.

Methods of the invention are particularly useful as POC tests for screening for HCV infection. In particular, methods of the invention can be carried out rapidly, without use of laboratory facilities or thermal cyclers. HCV infection can be detected within 1-2 weeks of infection. Once a subject has been identified as being infected with HCV, they can be administered appropriate treatment, and the infection can be monitored. If appropriate, the subject can be tested again to determine which HCV genotype is responsible for the infection, and then administered treatment appropriate to that genotype.

There is also provided according to the invention a kit for determining whether a sample includes HCV nucleic acid, which comprises:
a forward nucleic acid amplification primer and a reverse nucleic acid amplification primer, for amplifying a template nucleic acid by an isothermal amplification reaction, wherein each nucleic acid amplification primer hybridises specifically to HCV core nucleic acid sequence, or the complement thereof, that is conserved between at least HCV genotypes 1-6;
a nucleic acid capture probe, wherein the capture probe hybridises specifically to HCV core nucleic acid sequence, or the complement thereof, that is conserved between at least HCV genotypes 1-6; and/or
a nucleic acid detector probe, wherein the detector probe hybridises specifically to HCV core nucleic acid sequence, or the complement thereof, that is conserved between at least HCV genotypes 1-6, optionally wherein the detector probe comprises a visually detectable label for labelling a product of the isothermal nucleic acid amplification.

Optionally the forward nucleic acid primer comprises a nucleic acid sequence comprising or consisting of nucleic acid sequence: AGACTGCTAGCCGAGTAG (SEQ ID NO:1), or a nucleic acid sequence that has at least 90%, 91%, 92%, 93%, 94%, 95%, 96%, 97%, 98%, or 99% identity along its entire length with a nucleic acid sequence of SEQ ID NO:1.

Optionally the reverse nucleic acid primer comprises a nucleic acid sequence comprising or consisting of nucleic acid sequence: GCTCATGATGCACGGTCTACGAGA (SEQ ID NO:2), or a nucleic acid sequence that has at least 90%, 91%, 92%, 93%, 94%, 95%, 96%, 97%, 98%, or 99% identity along its entire length with a nucleic acid sequence of SEQ ID NO:2.

Optionally the capture probe comprises a nucleic acid sequence comprising or consisting of nucleic acid sequence: GCGAAAGGCCTTGTGGTACT (SEQ ID NO:3), or a nucleic acid sequence that has at least 90%, 91%, 92%, 93%, 94%, 95%, 96%, 97%, 98%, or 99% identity along its entire length with a nucleic acid sequence of SEQ ID NO:3, or the complement thereof.

Optionally the detector probe comprises a nucleic acid sequence comprising or consisting of nucleic acid sequence: TGATAGGGTGCTTGCGAGTG (SEQ ID NO:4), or a nucleic acid sequence that has at least 90%, 91%, 92%, 93%, 94%, 95%, 96%, 97%, 98%, or 99% identity along its entire length with a nucleic acid sequence of SEQ ID NO:4, or the complement thereof.

Optionally a kit of the invention further comprises an RNA-dependent DNA polymerase, a DNA-dependent DNA polymerase, a DNA/RNA duplex-specific ribonuclease, and a DNA-dependent RNA polymerase.

Optionally a kit of the invention further comprises appropriate nucleotide triphosphates (for transcription-based amplifications ribonucleotide triphosphates (rNTPs, i.e. rATP, rGTP, rCTP, and rUTP), and deoxyribonucleotide triphosphates (dNTPs, i.e. dATP, dGTP, dCTP, and dTTP) are required), a suitable buffer for carrying out the amplification reaction, and any necessary cofactors (for example magnesium ions) required by the enzyme activities. Examples of suitable buffers include Tris-HCl, HEPES, or acetate buffer. A suitable salt may be provided, such as potassium chloride or sodium chloride. Suitable concentrations of these components may readily be determined by the skilled person. Suitable rNTP concentrations are typically in the range 0.25-5 mM, or 0.5-2.5 mM. Suitable dNTP concentrations are typically in the range 0.25-5 mM dNTP, or 0.5-2.5 mM. Suitable magnesium ion concentrations are typically in the range 5-15 mM.

A kit of the invention may further comprise a visually detectable label for labelling a product of the isothermal nucleic acid amplification and/or a chromatographic test strip and reagents for capturing and detecting a product of the isothermal nucleic acid amplification. Suitable labels, test strips, and reagents, and methods for capturing and detecting a product of the isothermal nucleic acid amplification by a simple amplification-based assay (SAMBA), are described in WO 2008/090340 and Lee et al., Journal of Infectious Diseases 2010; 201(S1):S65-S71.

A kit of the invention may further comprise reagents for isolating nucleic acid from a sample, for example using a method of nucleic acid extraction as described above. Suitable reagents for extracting nucleic acid may include a lysis buffer for lysing cells present in the sample, a solid phase for binding nucleic acid, a binding buffer for binding nucleic acid to the solid phase (optionally, the lysis buffer is the same as the binding buffer) optionally a wash buffer for washing nucleic acid bound to the solid phase, and an elution buffer for eluting nucleic acid from the solid phase. Suitable lysis, wash, and elution buffers are described above, as well as suitable solid phases for use with the buffers.

A kit of the invention may further comprise any of the following additional components: a lancet for obtaining a sample of whole blood from a subject by finger prick or heel prick; a blood collector for collecting a sample of blood from a subject; positive and/or negative controls; instructions for carrying out a method of the invention testing using the kit.

There is also provided according to the invention a set of primers for amplifying HCV nucleic acid by an isothermal nucleic acid amplification reaction, which comprises a forward nucleic acid amplification primer and a reverse nucleic acid amplification primer, wherein each nucleic acid amplification primer hybridises specifically to HCV core nucleic acid sequence, or the complement thereof, that is conserved between at least HCV genotypes 1-6.

Optionally the set of primers comprises a forward nucleic acid primer comprising a nucleic acid sequence comprising or consisting of nucleic acid sequence: AGACTGCTAGCCGAGTAG (SEQ ID NO:1), or a nucleic acid sequence that has at least 90%, 91%, 92%, 93%, 94%, 95%, 96%, 97%, 98%, or 99% identity along its entire length with a nucleic acid sequence of SEQ ID NO:1.

Optionally the set of primers comprises a reverse nucleic acid primer comprising a nucleic acid sequence comprising or consisting of nucleic acid sequence: GCTCATGATGCACGGTCTACGAGA (SEQ ID NO:2), or a nucleic acid sequence that has at least 90%, 91%, 92%, 93%, 94%, 95%, 96%, 97%, 98%, or 99% identity along its entire length with a nucleic acid sequence of SEQ ID NO:2.

Optionally the forward and/or the reverse nucleic acid primer is up to 50 nucleotides long.

There is also provided according to the invention a set of oligonucleotides for amplifying HCV nucleic acid by an isothermal amplification reaction, and for capturing and/or detecting a product of the amplification reaction, which comprises:
a set of primers of the invention;
a nucleic acid capture probe, wherein the capture probe hybridises specifically to HCV core nucleic acid sequence, or the complement thereof, that is conserved between at least HCV genotypes 1-6; and/or
a nucleic acid detector probe, wherein the detector probe hybridises specifically to HCV core nucleic acid sequence, or the complement thereof, that is conserved between at least HCV genotypes 1-6.

Optionally the set of oligonucleotides comprises a forward nucleic acid primer comprising a nucleic acid sequence comprising or consisting of nucleic acid sequence: AGACTGCTAGCCGAGTAG (SEQ ID NO:1), or a nucleic acid sequence that has at least 90%, 91%, 92%, 93%, 94%, 95%, 96%, 97%, 98%, or 99% identity along its entire length with a nucleic acid sequence of SEQ ID NO:1.

Optionally the set of oligonucleotides comprises a reverse nucleic acid primer comprising a nucleic acid sequence comprising or consisting of nucleic acid sequence: GCTCATGATGCACGGTCTACGAGA (SEQ ID NO:2), or a nucleic acid sequence that has at least 90%, 91%, 92%, 93%, 94%, 95%, 96%, 97%, 98%, or 99% identity along its entire length with a nucleic acid sequence of SEQ ID NO:2.

Optionally the set of oligonucleotides comprises a capture probe comprising a nucleic acid sequence comprising or consisting of nucleic acid sequence: GCGAAAGGCCTTGTGGTACT (SEQ ID NO:3), or a nucleic acid sequence that has at least 90%, 91%, 92%, 93%, 94%, 95%, 96%, 97%, 98%, or 99% identity along its entire length with a nucleic acid sequence of SEQ ID NO:3, or the complement thereof.

Optionally the set of oligonucleotides comprises a detector probe comprising a nucleic acid sequence comprising or consisting of nucleic acid sequence: TGATAGGGTGCTTGCGAGTG (SEQ ID NO:4), or a nucleic acid sequence that has at least 90%, 91%, 92%, 93%, 94%, 95%, 96%, 97%, 98%, or 99% identity along its entire length with a nucleic acid sequence of SEQ ID NO:4, or the complement thereof.

Optionally the capture and/or detector probe is up to 50 nucleotides long.

There is also provided according to the invention an oligonucleotide, which comprises:
a nucleic acid sequence comprising or consisting of nucleic acid sequence: AGACTGCTAGCCGAGTAG (SEQ ID NO:1), or a nucleic acid sequence that has at least 90%, 91%, 92%, 93%, 94%, 95%, 96%, 97%, 98%, or 99% identity along its entire length with a nucleic acid sequence of SEQ ID NO:1, or the complement thereof;
a nucleic acid sequence comprising or consisting of nucleic acid sequence: GCTCATGATGCACGGTCTACGAGA (SEQ ID NO:2), or a nucleic acid sequence that has at least 90%, 91%, 92%, 93%, 94%, 95%, 96%, 97%, 98%, or 99% identity along its entire length with a nucleic acid sequence of SEQ ID NO:2, or the complement thereof;
a nucleic acid sequence comprising or consisting of: nucleic acid sequence GCGAAAGGCCTTGTGGTACT (SEQ ID NO:3), or a nucleic acid sequence that has at least 90%, 91%, 92%, 93%, 94%, 95%, 96%, 97%, 98%, or 99% identity along its entire length with a nucleic acid sequence of SEQ ID NO:3, or the complement thereof; or
a nucleic acid sequence comprising or consisting of: nucleic acid sequence TGATAGGGTGCTTGCGAGTG (SEQ ID NO:4), or a nucleic acid sequence that has at least 90%, 91%, 92%, 93%, 94%, 95%, 96%, 97%, 98%, or 99% identity along its entire length with a nucleic acid sequence of SEQ ID NO:4, or the complement thereof.

A set of primers, a set of oligonucleotides, or an oligonucleotide, of the invention may be used in a kit of the invention, or in a method of the invention.

A primer, probe, or oligonucleotide of the invention, or of a set of primers or oligonucleotides of the invention, or of a kit of the invention, or for use in a method of the invention, may be at least 15, 20, 25, 30, 35, 40, 45, 50, or over 50 nucleotides in length.

A primer, probe, or oligonucleotide of the invention, or of a set of primers or oligonucleotides of the invention, or of a kit of the invention, or for use in a method of the invention, may be up to 20, 25, 30, 35, 40, 45, 50, or 100 nucleotides in length.

A primer or oligonucleotide of the invention, or of a set of primers or oligonucleotides of the invention, or of a kit of the invention, or for use in a method of the invention, which comprises a nucleic acid sequence: AGACTGCTAGCCGAGTAG (SEQ ID NO:1) may be up to 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 nucleotides in length.

An oligonucleotide of the invention, or of a set of primers or oligonucleotides of the invention, or of a kit of the invention, or for use in a method of the invention, which comprises the complement of a nucleic acid sequence: AGACTGCTAGCCGAGTAG (SEQ ID NO:1) may be up to 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 nucleotides in length.

A primer or oligonucleotide of the invention, or of a set of primers or oligonucleotides of the invention, or of a kit of the invention, or for use in a method of the invention, which comprises a nucleic acid sequence: GCTCATGATGCACGGTCTACGAGA (SEQ ID NO:2) may be up to 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 nucleotides in length.

An oligonucleotide of the invention, or of a set of primers or oligonucleotides of the invention, or of a kit of the invention, or for use in a method of the invention, which comprises the complement of a nucleic acid sequence: GCTCATGATGCACGGTCTACGAGA (SEQ ID NO:2) may be up to 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 nucleotides in length.

A probe or oligonucleotide of the invention, or of a set of primers or oligonucleotides of the invention, or of a kit of the invention, or for use in a method of the invention, which comprises a nucleic acid sequence: GCGAAAGGCCTTGTGGTACT (SEQ ID NO:3) may be up to 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 nucleotides in length.

A probe or oligonucleotide of the invention, or of a set of primers or oligonucleotides of the invention, or of a kit of the invention, or for use in a method of the invention, which comprises the complement of a nucleic acid sequence: GCGAAAGGCCTTGTGGTACT (SEQ ID NO:3) may be up to 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 nucleotides in length.

A probe or oligonucleotide of the invention, or of a set of primers or oligonucleotides of the invention, or of a kit of the invention, or for use in a method of the invention, which comprises a nucleic acid sequence: TGATAGGGTGCTTGCGAGTG (SEQ ID NO:4) may be up to 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 nucleotides in length.

A probe or oligonucleotide of the invention, or of a set of primers or oligonucleotides of the invention, or of a kit of the invention, or for use in a method of the invention, which comprises the complement of a nucleic acid sequence: TGATAGGGTGCTTGCGAGTG (SEQ ID NO:4) may be up to 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 nucleotides in length.

An oligonucleotide of the invention may comprise a nucleotide sequence that comprises or consists of a sequence that is at least 90%, 91%, 92%, 93%, 94%, 95%, 96%, 97%, 98%, or 99% identical, or that is 100% identical, over its entire length to the nucleotide sequence of any of SEQ ID NOs: 1-4, or 7, or the complement thereof.

An oligonucleotide of the invention may comprise a nucleotide sequence that comprises or consists of a sequence that is at least 90%, 91%, 92%, 93%, 94%, 95%, 96%, 97%, 98%, or 99% identical, or that is 100% identical, over its entire length to the nucleotide sequence of SEQ ID NO: 7, or the complement thereof.

The oligonucleotide may be labelled, for example with a visually detectable label. In particular, an oligonucleotide that comprises or consists of a nucleic acid sequence of: TGATAGGGTGCTTGCGAGTG (SEQ ID NO:4), or a nucleic acid sequence that has at least 90%, 91%, 92%, 93%, 94%, 95%, 96%, 97%, 98%, or 99% identity along its entire length with a nucleic acid sequence of SEQ ID NO:4, or the complement thereof, may be labelled with a visually detectable label. Examples of visually detectable labels include colloidal metal sol particles, latex particles, or textile dye particles. An example of colloidal metal sol particles is colloidal gold particles.

A set of primers or oligonucleotides of the invention may comprise an oligonucleotide of the invention.

A kit of the invention may comprise a set of primers, a set of oligonucleotides, or an oligonucleotide, of the invention.

There is also provided according to the invention use of a set of primers, a set of oligonucleotides, or an oligonucleotide, of the invention in a method of the invention.

Embodiments of the invention are described below, by way of example only, with reference to the accompanying drawings in which:

FIG. 1 shows the structure of the HCV RNA genome;

FIG. 2 shows nucleic acid sequence of the HCV core region, and nucleic acid sequence of HCV primers and probes according to an embodiment of the invention. The corresponding sequence of the primers and probes in the HCV core sequence are shown underlined in bold;

FIG. 3 shows a nucleic acid sequence alignment of HCV core region of HCV genotypes 1-6, and the locations in the core sequence of primer and probe sequences according to embodiments of the invention. The sequences shown in the alignment (and their respective SEQ ID NOs) are:

(SEQ ID NO: 8)
HCV_1_Spain_AJ8;
(SEQ ID NO: 9)
HCV_1a_USA_EF40;
(SEQ ID NO: 10)
HCV_1b_Japan_AB;
(SEQ ID NO: 11)
HCV_1b/2b_Japan;
(SEQ ID NO: 12)
HCV_1b/2k_Russi;
(SEQ ID NO: 13)
HCV_1c_Indonesi;
(SEQ ID NO: 14)
HCV_1e_UK_KC248;
(SEQ ID NO: 15)
HCV_1f_France_L;
(SEQ ID NO: 16)
HCV_1g_Spain_AM;
(SEQ ID NO: 17)
HCV_1h_Cameroon;
(SEQ ID NO: 18)
HCV_1I_UK_KC248;
(SEQ ID NO: 19)
HCV_2a_Japan_B0;
(SEQ ID NO: 20)
HCV_2b_AB030907;
(SEQ ID NO: 21)
HCV_2b/1b_Phili;
(SEQ ID NO: 22)
HCV_2k_Moldova;
(SEQ ID NO: 23)
HCV_2/5_France;
(SEQ ID NO: 24)
HCV_3a_AB691595;
(SEQ ID NO: 25)
HCV_3a_AB691596;
(SEQ ID NO: 26)
HCV_3a_AF046866;
(SEQ ID NO: 27)
HCV_3h_Somalia;
(SEQ ID NO: 28)
HCV_4_USA_DQ418;
(SEQ ID NO: 29)
HCV_4a_Egypt_DQ;
(SEQ ID NO: 30)
HCV_4a_Japan_AB;
(SEQ ID NO: 31)
HCV_4b_Portugal;
(SEQ ID NO: 32)
HCV_4c_Canada_F;
(SEQ ID NO: 33)
HCV_4d_USA_DQ41;
(SEQ ID NO: 34)
HCV_4d_Spain_DQ;
(SEQ ID NO: 35)
HCV_4f_France_E;
(SEQ ID NO: 36)
HCV_4g_Canada_F;
(SEQ ID NO: 37)
HCV_4I_Canada_F;
(SEQ ID NO: 38)
HCV_4k_EU392171;
(SEQ ID NO: 39)
HCV_4m_Canada_F;
(SEQ ID NO: 40)
HCV_4n_Canada_F;
(SEQ ID NO: 41)
HCV_4i_Canada_F;
(SEQ ID NO: 42)
HCV_4p_Canada_F;
(SEQ ID NO: 43)
HCV_4q_Canada_F;
(SEQ ID NO: 44)
HCV_4r_Canada_F;
(SEQ ID NO: 45)
HCV_4t_Canada_F;
(SEQ ID NO: 46)
HCV_4v_Cyprus_H;
(SEQ ID NO: 47)
HCV_5_India_KF3;
(SEQ ID NO: 48)
HCV_5a_China_KC;
(SEQ ID NO: 49)
HCV_5a_France_A;
(SEQ ID NO: 50)
HCV_5a_UK_NC_00;
(SEQ ID NO: 51)
HCV_6_China_DQ2;
(SEQ ID NO: 52)
HCV_6a_HongKong;
(SEQ ID NO: 53)
HCV_6b_Thailand;
(SEQ ID NO: 54)
HCV_6c_Thailand;
(SEQ ID NO: 55)
HCV_6d_Vietnam;
(SEQ ID NO: 56)
HCV_6e_China_DQ;
(SEQ ID NO: 57)
HCV_6f_Thailand;
(SEQ ID NO: 58)
HCV_6g_HongKong;
(SEQ ID NO: 59)
HCV_6h_Vietnam;
(SEQ ID NO: 60)
HCV_6i_Thailand;
(SEQ ID NO: 61)
HCV_6k_China_DQ;
(SEQ ID NO: 62)
HCV_6l_Vietnam;

FIG. 4 shows schematically the steps for transcription-based amplification of a target RNA; and

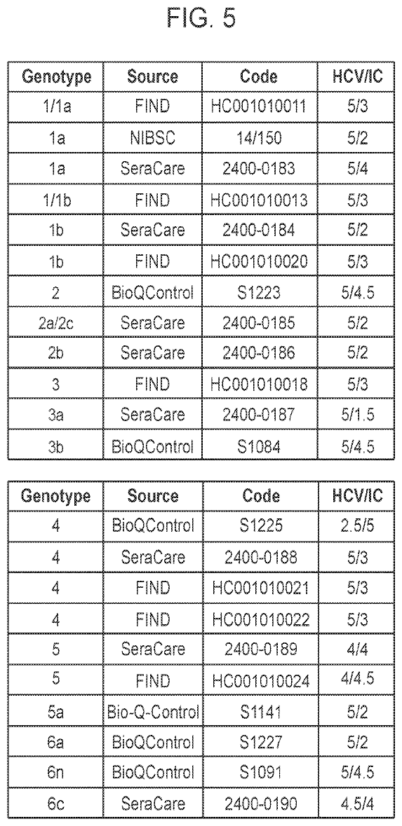

FIG. 5 shows detection of HCV genotypes 1-6 using a method according to an embodiment of the invention. HCV subtypes were tested at 3,000 IU/ml in whole blood. At least 3 plasma samples of each subtype diluted in whole blood were detected at 3,000 IU/ml.

### EXAMPLE

### Point-of-Care (POC) Nucleic Acid Test for Detecting HCV Genotypes 1-6

HCV viral RNA was extracted, reverse transcribed, and amplified by isothermal nucleic acid amplification, and the amplification products were detected by rapid visual detection with a dipstick, using a simple amplification-based assay (SAMBA) method similar to the method described in Lee et al., Journal of Infectious Diseases 2010; 201(S1):S65-S71.

Briefly, a reverse nucleic acid amplification primer comprises nucleic acid sequence complementary to a portion of HCV target RNA so that the primer can hybridise specifically to the target RNA, and a single stranded-version of a promoter sequence for a DNA-dependent RNA polymerase at its 5′-end. The reverse primer hybridizes to the RNA target. An RNA-dependent DNA polymerase extends the reverse primer to synthesise a complementary DNA (cDNA) copy of the RNA target. A DNA/RNA duplex-specific ribonuclease digests the RNA of the RNA-cDNA hybrid. A forward nucleic acid amplification primer comprises nucleic acid sequence complementary to a portion of the cDNA. The forward primer hybridizes to the cDNA downstream of the part of the cDNA formed by the reverse primer. The forward primer is extended by a DNA-dependent DNA polymerase to produce a second DNA strand which extends through the DNA-dependent RNA polymerase promoter sequence at one end (thereby forming a double stranded promoter). This promoter is used by a DNA-dependent RNA polymerase to synthesise a large number of RNAs complementary to the original target sequence. These RNA products then function as templates for a cyclic phase of the reaction, but with the primer hybridising steps reversed, i.e., the forward primer followed by the reverse primer.

The following primer sequences were used for isothermal amplification of HCV nucleic acid of genotypes 1-6:
HCV Primer F2 (forward primer):

(SEQ ID NO: 1)
GACTGCTAGCCGAGTAG;

HCV Primer REV1.2 (reverse primer)/T7 promoter:

(SEQ ID NO: 7)
GCTCATGATGCACGGTCTACGAGATAATACGACTCACTATAG.

Amplification product was captured and detected using the following capture and detection probes:
HCV Probe CP2 (capture probe):

(SEQ ID NO: 3)
GCGAAAGGCCTTGTGGTACT;

HCV Probe DP2 (detector probe):

(SEQ ID NO: 4)
TGATAGGGTGCTTGCGAGTG.

The HCV primers/probes were tested against at least three different samples for each of the six HCV genotypes. The results are recorded in FIG. 5. HCV genotypes 1-6 were efficiently detected.

1. A method for determining whether a sample includes HCV nucleic acid, which comprises amplifying nucleic acid of the sample, or amplifying nucleic acid derived from nucleic acid of the sample, by an isothermal amplification reaction using a forward nucleic acid amplification primer and a reverse nucleic acid amplification primer, wherein each nucleic acid amplification primer hybridises specifically to HCV core nucleic acid sequence, or the complement thereof, that is conserved between at least HCV genotypes 1-6; wherein:
the forward nucleic acid primer comprises a nucleic acid sequence of: AGACTGCTAGCCGAGTAG (SEQ ID NO:1), or a nucleic acid sequence that has at least 90%, 95%, 96%, 97%, 98%, or 99% identity along its entire length with a nucleic acid sequence of SEQ ID NO:1; and
the reverse nucleic acid primer comprises a nucleic acid sequence of: GCTCATGATGCACGGTCTACGAGA (SEQ ID NO:2), or a nucleic acid sequence that has at least 95%, 96%, 97%, 98%, or 99% identity along its entire length with a nucleic acid sequence of SEQ ID NO:2.

2.-3. (canceled)

4. The method according to claim 1, which further comprises reverse transcribing HCV RNA of the sample, and amplifying a product of the reverse transcription by an isothermal amplification reaction using the forward and reverse nucleic acid amplification primers.

5. The method according to claim 4, wherein the reverse nucleic acid primer further comprises a promoter sequence for a DNA-dependent RNA polymerase at its 5′-end, and reverse transcription is carried out using the reverse nucleic acid amplification primer.

6. The method according to claim 4, which further comprises isolating a nucleic acid of the sample before reverse transcribing HCV RNA of the sample present in the isolated nucleic acid.

7. The method according to claim 1, which further comprises capturing a product of the isothermal amplification reaction by hybridising a nucleic acid of the product to a nucleic acid capture probe, wherein the capture probe hybridises specifically to HCV core nucleic acid sequence, or the complement thereof, that is conserved between at least HCV genotypes 1-6.

8. The method according to claim 7, wherein the capture probe comprises a nucleic acid sequence of: GCGAAAGGCCTTGTGGTACT (SEQ ID NO:3), or a nucleic acid sequence that has at least 90%, 91%, 92%, 93%, 94%, 95%, 96%, 97%, 98%, or 99% identity along its entire length with a nucleic acid sequence of SEQ ID NO:3, or the complement thereof.

9. The method according to claim 1, which further comprises detecting a product of the isothermal amplification reaction by hybridising the product to a nucleic acid detector probe, wherein the detector probe hybridises specifically to HCV core nucleic acid sequence, or the complement thereof, that is conserved between at least HCV genotypes 1-6.

10. The method according to claim 9, wherein the detector probe comprises a nucleic acid sequence of: TGATAGGGTGCTTGCGAGTG (SEQ ID NO:4), or a nucleic acid sequence that has at least 90%, 91%, 92%, 93%, 94%, 95%, 96%, 97%, 98%, or 99% identity along its entire length with a nucleic acid sequence of SEQ ID NO:4, or the complement thereof.

11. The method according to claim 9, wherein the detector probe is labelled with a visually detectable label.

12. The method according to claim 7, wherein capture and/or detection of the product of the isothermal amplification reaction is carried out by chromatographic dipstick assay.

13. The method according to claim 1, wherein the sample is a biological sample obtained from a subject suspected of being infected with HCV.

14. The method according to claim 1, wherein the sample is a blood or a plasma sample obtained from a subject suspected of being infected with HCV.

15. The method according to claim 1 which is an in vitro method.

16. A kit for determining whether a sample includes HCV nucleic acid, which comprises:
a forward nucleic acid amplification primer and a reverse nucleic acid amplification primer, for amplifying a template nucleic acid by an isothermal amplification reaction, wherein each nucleic acid amplification primer hybridises specifically to HCV core nucleic acid sequence, or the complement thereof, that is conserved between at least HCV genotypes 1-6;
a nucleic acid capture probe, wherein the capture probe hybridises specifically to HCV core nucleic acid sequence, or the complement thereof, that is conserved between at least HCV genotypes 1-6; and/or
a nucleic acid detector probe, wherein the detector probe hybridises specifically to HCV core nucleic acid sequence, or the complement thereof, that is conserved between at least HCV genotypes 1-6, optionally wherein the detector probe comprises a visually detectable label for labelling a product of the isothermal nucleic acid amplification; wherein:
the forward nucleic acid primer comprises a nucleic acid sequence of: AGACTGCTAGCCGAGTAG (SEQ ID NO:1), or a nucleic acid sequence that has at least 95%, 96%, 97%, 98%, or 99% identity along its entire length with a nucleic acid sequence of SEQ ID NO:1, and
the reverse nucleic acid primer comprises a nucleic acid sequence of: GCTCATGATGCACGGTCTACGAGA (SEQ ID NO:2), or a nucleic acid sequence that has at least 95%, 96%, 97%, 98%, or 99% identity along its entire length with a nucleic acid sequence of SEQ ID NO:2.

17.-18. (canceled)

19. The kit according to claim 16, wherein the capture probe comprises a nucleic acid sequence of: GCGAAAGGCCTTGTGGTACT (SEQ ID NO:3), or a nucleic acid sequence that has at least 90%, 91%, 92%, 93%, 94%, 95%, 96%, 97%, 98%, or 99% identity along its entire length with a nucleic acid sequence of SEQ ID NO:3, or the complement thereof.

20. The kit according to claim 16, wherein the detector probe comprises a nucleic acid sequence of: TGATAGGGTGCTTGCGAGTG (SEQ ID NO:4), or a nucleic acid sequence that has at least 90%, 91%, 92%, 93%, 94%, 95%, 96%, 97%, 98%, or 99% identity along its entire length with a nucleic acid sequence of SEQ ID NO:4, or the complement thereof.

21. The kit according to claim 16, which further comprises an RNA-dependent DNA polymerase, a DNA-dependent DNA polymerase, a DNA/RNA duplex-specific ribonuclease, and a DNA-dependent RNA polymerase.

22. The kit according to claim 16, which further comprises a lancet for obtaining a sample of whole blood from a subject by finger prick or heel prick.

23. The kit according to claim 16, which further comprises a blood collector for collecting a sample of blood from a subject.

24. The kit according to claim 16, which further comprises a chromatographic test strip for capturing and detecting a product of the isothermal nucleic acid amplification.

25. The kit according to claim 16, which further comprises a lysis/binding buffer, an elution buffer, and optionally a wash buffer, for extracting nucleic acid from a blood or plasma sample.

26. A set of primers for amplifying HCV nucleic acid by an isothermal nucleic acid amplification reaction, which comprises a forward nucleic acid amplification primer and a reverse nucleic acid amplification primer, wherein each nucleic acid amplification primer hybridises specifically to HCV core nucleic acid sequence, or the complement thereof, that is conserved between at least HCV genotypes 1-6; wherein:
the forward nucleic acid primer comprises a nucleic acid sequence of: AGACTGCTAGCCGAGTAG (SEQ ID NO:1), or a nucleic acid sequence that has at least 95%, 96%, 97%, 98%, or 99% identity along its entire length with a nucleic acid sequence of SEQ ID NO:1, and
the reverse nucleic acid primer comprises a nucleic acid sequence of: GCTCATGATGCACGGTCTACGAGA (SEQ ID NO:2), or a nucleic acid sequence that has at least 95%, 96%, 97%, 98%, or 99% identity along its entire length with a nucleic acid sequence of SEQ ID NO:2.

27.-28. (canceled)

29. The set of primers according to claim 26, wherein the forward and/or the reverse nucleic acid primer is up to 50 nucleotides long.

30. A set of oligonucleotides for amplifying HCV nucleic acid by an isothermal nucleic acid amplification reaction, and for capturing and/or detecting a product of the amplification reaction, which comprises:
the set of primers according to claim 26;
a nucleic acid capture probe, wherein the capture probe hybridises specifically to HCV core nucleic acid sequence, or the complement thereof, that is conserved between at least HCV genotypes 1-6; and/or
a nucleic acid detector probe, wherein the detector probe hybridises specifically to HCV core nucleic acid sequence, or the complement thereof, that is conserved between at least HCV genotypes 1-6.

31.-32. (canceled)

33. The set of oligonucleotides according to claim 30, wherein the capture probe comprises a nucleic acid sequence of:
GCGAAAGGCCTTGTGGTACT (SEQ ID NO:3), or a nucleic acid sequence that has at least 90%, 91%, 92%, 93%, 94%, 95%, 96%, 97%, 98%, or 99% identity along its entire length with a nucleic acid sequence of SEQ ID NO:3, or the complement thereof.

34. The set of oligonucleotides according to claim 30, wherein the detector probe comprises a nucleic acid sequence of:
TGATAGGGTGCTTGCGAGTG (SEQ ID NO:4), or a nucleic acid sequence that has at least 90%, 91%, 92%, 93%, 94%, 95%, 96%, 97%, 98%, or 99% identity along its entire length with a nucleic acid sequence of SEQ ID NO:4, or the complement thereof.

35. The set of oligonucleotides according to claim 30, wherein the capture and/or detector probe is up to 50 nucleotides long.

36.-39. (canceled)


SEQUENCE LISTING (62 sequences)

SEQ ID NO: 1 - DNA, 18 bp, synthetic construct
  misc_feature 1..18: note = Forward primer
  source 1..18: mol_type = other DNA
agactgctagccgagtag

SEQ ID NO: 2 - DNA, 24 bp, synthetic construct
  misc_feature 1..24: note = Reverse primer
  source 1..24: mol_type = other DNA
gctcatgatgcacggtctacgaga

SEQ ID NO: 3 - DNA, 20 bp, synthetic construct
  misc_feature 1..20: note = Capture probe
  source 1..20: mol_type = other DNA
gcgaaaggccttgtggtact

SEQ ID NO: 4 - DNA, 20 bp, synthetic construct
  misc_feature 1..20: note = Detector probe
  source 1..20: mol_type = other DNA
tgatagggtgcttgcgagtg

SEQ ID NO: 5 - DNA, 1140 bp, Hepatitis C Virus
  source 1..1140: mol_type = other DNA
gtgaggaactactgtcttcacgcagaaagcgtctagccatggcgttagtatgagtgtcgtacagcctccaggcccccccctcccgggagagccatagtggtctgcggaaccggtgagtacaccggaattgccaggatgaccgggtcctttccattggatcaaacccgctcaatgcctggagatttgggcgtgcccccgcaagactgctagccgagtagcgttgggttgcgaaaggccttgtggactgcctgatagggtgcttgcgagtgccccgggaggtctcgtagaccgtgcatcatgagcacacttccaaaacctcaaagaaaaaccaaaagaaacaccaaccgccgcccaatggacgtcaagttcccgggtggcggtcagatcgttggtgaggaactactgtcttcacgcagaaagcgtctagccatggcgttagtatgagtgtcgtacagcctccaggcccccccctcccgggagagccatagtggtctgcggaaccggtgagtacaccggaattgccaggatgaccgggtcctttccattggatcaaacccgctcaatgcctggagatttgggcgtgcccccgcaagactgctagccgagtagcgttgggttgcgaaaggccttgtggtactgcctgatagggtgcttgcgagtgccccgggaggtctcgtagaccgtgcatcatgagcacacttccaaaacctcaaagaaaaaccaaaagaaacaccaaccgccgcccaatggacgtcaagttcccgggtggcggtcagatcgttggtgaggaactactgtcttcacgcagaaagcgtctagccatggcgttagtatgagtgtcgtacagcctccaggcccccccctcccgggagagccatagtggtctgcggaaccggtgagtacaccggaattgccaggatgaccgggtcctttccattggatcaaacccgctcaatgcctggagatttgggcgtgcccccgcaagactgctagccgagtagcgttgggttgcgaaaggccttgtggactgcctgatagggtgcttgcgagtgccccgggaggtctcgtagaccgtgcatcatgagcacacttccaaaacctcaaagaaaaaccaaaagaaacaccaaccgccgcccaatg

SEQ ID NO: 6 - DNA, 18 bp, synthetic construct
  misc_feature 1..18: note = T7 promoter sequence
  source 1..18: mol_type = other DNA
taatacgactcactatag

SEQ ID NO: 7 - DNA, 42 bp, synthetic construct
  misc_feature 1..42: note = Reverse primer with T7 promoter sequence
  source 1..42: mol_type = other DNA
gctcatgatgcacggtctacgagataatacgactcactatag

SEQ ID NO: 8 - DNA, 198 bp, Hepatitis C Virus
  source 1..198: mol_type = other DNA
ttggatcaaacccgctctatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 9 - DNA, 197 bp, Hepatitis C Virus
  source 1..197: mol_type = other DNA
ttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcg

SEQ ID NO: 10 - DNA, 198 bp, Hepatitis C Virus
  source 1..198: mol_type = other DNA
ttggatcaatcccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacaaatcctaaacctcaaagaaaaaccaaacgtaacaccaaccgccg

SEQ ID NO: 11 - DNA, 197 bp, Hepatitis C Virus
  source 1..197: mol_type = other DNA
ttggataaacccactctatgtccggtcatttgggcgtgcccccgcaagactgctagccgagtagcgttgggttgcgaaaggccttgtggtactgcctgatagggtgcttgcgagtgccccgggaggtctcgtagaccgtgcatcatgagcacaaatcctaaacctcaaagaaaaacccaaagaaacacaaaccgccg

SEQ ID NO: 12 - DNA, 197 bp, Hepatitis C Virus
  source 1..197: mol_type = other DNA
ttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcatcatgagcacaaatcctaaacctcaaagaaaaaccaaaagaaacacaaaccgccg

SEQ ID NO: 13 - DNA, 197 bp, Hepatitis C Virus
  source 1..197: mol_type = other DNA
ttggattaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 14 - DNA, 197 bp, Hepatitis C Virus
  source 1..197: mol_type = other DNA
ttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaaagaaacaccaaccgccg

SEQ ID NO: 15 - DNA, 197 bp, Hepatitis C Virus
  source 1..197: mol_type = other DNA
ttggatctaaccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacgaatcctaaacctcaaagaaaaaccaaacgcaacaccaaccgccg

SEQ ID NO: 16 - DNA, 197 bp, Hepatitis C Virus
  source 1..197: mol_type = other DNA
ttggataaacccgctcaatgcctggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaaagaaacaccaaccgccg

SEQ ID NO: 17 - DNA, 197 bp, Hepatitis C Virus
  source 1..197: mol_type = other DNA
ttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacaaatcctaaacctcaaagaaaaaccaaacgtaacaccaaccgccg

SEQ ID NO: 18 - DNA, 198 bp, Hepatitis C Virus
  source 1..198: mol_type = other DNA
ttggatcaaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcg

SEQ ID NO: 19 - DNA, 197 bp, Hepatitis C Virus
  source 1..197: mol_type = other DNA
ttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacaaatcctaaacctcaaagaaaaaccaaaagaaacaccaaccgtcg

SEQ ID NO: 20 - DNA, 197 bp, Hepatitis C Virus
  source 1..197: mol_type = other DNA
ttggataaacccactctatgtccggtcatttgggcgtgcccccgcaagactgctagccgagtagcgttgggttgcgaaaggccttgtggtactgcctgatagggtgcttgcgagtgccccgggaggcctcgtagaccgtgcaccatgagcacaaatcctaaacctcaaagaaaaaccaaaagaaacacaaaccgccg

SEQ ID NO: 21 - DNA, 197 bp, Hepatitis C Virus
  source 1..197: mol_type = other DNA
ttggataaacccactctatgtccggtcatttgggcgtgcccccgcaagactgctagccgagtagcgttgggttgcgaaaggccttgtggtactgcctgatagggtgcttgcgagtgccccgggaggtctcgtagaccgtgcatcatgagcacagatcctaaaccccaaagaaaaaccaaaagaaacacaaaccgccg

SEQ ID NO: 22 - DNA, 197 bp, Hepatitis C Virus
  source 1..197: mol_type = other DNA
ttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacacaaaccgccg

SEQ ID NO: 23 - DNA, 197 bp, Hepatitis C Virus
  source 1..197: mol_type = other DNA
ttggataaacccactctatgcccggtcatttgggcgtgcccccgcaagactgctagccgagtagcgttgggttgcgaaaggccttgtggtactgcctgatagggtgcttgcgagtgccccgggaggtctcgtagaccgtgcatcatgagcacaaatcctaaacctcaaagaaaaaccaacagaaacactaaccgccg

SEQ ID NO: 24 - DNA, 197 bp, Hepatitis C Virus
  source 1..197: mol_type = other DNA
ttggaacaacccgctcaatacccagaaatttgggcgtgcccccgcgagatcactagccgagtagtgttgggtcgcgaaaggccttgtggtactgcctgatagggtgcttgcgagtgccccgggaggtctcgtagaccgtgcaacatgagcacacttcctaaaccccaaagaaaaaccaaaagaaacaccatccgtcg

SEQ ID NO: 25 - DNA, 197 bp, Hepatitis C Virus; identical to SEQ ID NO: 24
  source 1..197: mol_type = other DNA

SEQ ID NO: 26 - DNA, 197 bp, Hepatitis C Virus
  source 1..197: mol_type = other DNA
ttggaacaacccgctcaatacccagaaatttgggcgtgcccccgcgagatcactagccgagtagtgttgggtcgcgaaaggccttgtggtactgcctgatagggtgcttgcgagtgccccgggaggtctcgtagaccgtgcaacatgagcacacttcctaaaccacaaagaaaaaccaaaagaaacaccatccgtcg

SEQ ID NO: 27 - DNA, 53 bp, Hepatitis C Virus
  source 1..53: mol_type = other DNA
atgagcacacttcctaaacctcaaagaaaaaccaaaagaaacaccatccgccg

SEQ ID NO: 28 - DNA, 197 bp, Hepatitis C Virus
  source 1..197: mol_type = other DNA
ttggattaacccgctcactgcccggaaatttgggcgtgcccccgcaagactactagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaccgtaacaccaaccgccg

SEQ ID NO: 29 - DNA, 197 bp, Hepatitis C Virus
  source 1..197: mol_type = other DNA
ttggattaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 30 - DNA, 198 bp, Hepatitis C Virus
  source 1..198: mol_type = other DNA
ttggattaaacccgctcaatgcccggaaatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 31 - DNA, 197 bp, Hepatitis C Virus
  source 1..197: mol_type = other DNA
ttggatcaacccgctcaatacccggaaatttgggcgtgcccccgcaagacygctagccgagtagtgttgggtcgcgaaaggccttgtggtactgcctgatagggtgcttgcgagtgccccgggaggtctcgtagaccgtgcatcatgagcacaaatcctaaaccccaaagaaaaaccaaacgtaacaccaaccgtcg

SEQ ID NO: 32 - DNA, 197 bp, Hepatitis C Virus
  source 1..197: mol_type = other DNA
ttggataaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 33 - DNA, 197 bp, Hepatitis C Virus
  source 1..197: mol_type = other DNA
ttggataaacccgctcaatgcccggaaatttgggcgtgcccccgcaagactgctagccgagtagygttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 34 - DNA, 197 bp, Hepatitis C Virus
  source 1..197: mol_type = other DNA
ttggataaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaaccgcaaagaaaaacccaacgtaacaccaaccgccg

SEQ ID NO: 35 - DNA, 197 bp, Hepatitis C Virus
  source 1..197: mol_type = other DNA
ttggattaacccgctcaatgcccggagatttgggcgtgcccccgcaagactgctagccgagtagcgttgggttgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 36 - DNA, 198 bp, Hepatitis C Virus
  source 1..198: mol_type = other DNA
ttggaacaaacccgctcaatgcccggcaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 37 - DNA, 197 bp, Hepatitis C Virus
  source 1..197: mol_type = other DNA
ttggataaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaacccaccgtaacaccaaccgccg

SEQ ID NO: 38 - DNA, 198 bp, Hepatitis C Virus
  source 1..198: mol_type = other DNA
ttggaactaacccgctcaatgcccggaaatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagacaaaccaaacgtaacaccaaccgccg

SEQ ID NO: 39 - DNA, 198 bp, Hepatitis C Virus
  source 1..198: mol_type = other DNA
ttggatcaaacccgctcaatgcctggaaatttgggcgtgcccccgcaagactgctagccgagtagcgttgggttgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 40 - DNA, 197 bp, Hepatitis C Virus
  source 1..197: mol_type = other DNA
ttggatcaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcccaaacctcaaagaaaaaccaaacgtaacaccaaccgccg

SEQ ID NO: 41 - DNA, 198 bp, Hepatitis C Virus
  source 1..198: mol_type = other DNA
ttggaataaacccgctcaatgcccggcaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 42 - DNA, 197 bp, Hepatitis C Virus; identical to SEQ ID NO: 32
  source 1..197: mol_type = other DNA

SEQ ID NO: 43 - DNA, 198 bp, Hepatitis C Virus
  source 1..198: mol_type = other DNA
ttggaacaaacccgctcaatgcccggaaatttgggcgtgcccccgcaagactgctagccgagtagcgttgggttgcgaaaggccttgtggtactgcctgatagggtgcttgcgagtgccccgggaggtttcgtagaccgtgcaccatgagcacgaatcctaaacctcaaagaaaaaccaaacgtaacaccaaccgccg

SEQ ID NO: 44 - DNA, 197 bp, Hepatitis C Virus
  source 1..197: mol_type = other DNA
ttggattaacccgctcaatgcctggaaatttgggcgtgcccccgcgagactgctagccgagtagtgttgggtcgcgaaaggccttgtggtactgcctgatagggtgcttgcgagtgccccgggaggtctcgtagaccgtgcaccatgagcacaaatcccaaacctcaaagaaaaaccaaacgtaacaccaaccgccg

SEQ ID NO: 45 - DNA, 197 bp, Hepatitis C Virus; identical to SEQ ID NO: 29
  source 1..197: mol_type = other DNA

SEQ ID NO: 46 - DNA, 198 bp, Hepatitis C Virus
  source 1..198: mol_type = other DNA
ttggaataaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccg

SEQ ID NO: 47 - DNA, 197 bp, Hepatitis C Virus
  source 1..197: mol_type = other DNA
gtggataaacccgctcaatgcccggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaaagaaacaccaaccgtcg

SEQ ID NO: 48 - DNA, 197 bp, Hepatitis C Virus
  source 1..197: mol_type = other DNA
ttggataaacccgctcaatgcccggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaaagaaacaccaaccgccg

SEQ ID NO: 49 - DNA, 197 bp, Hepatitis C Virus
  source 1..197: mol_type = other DNA
ttggataaacccgctcaatgcccggagatttgggcgtgcccccgcgagactgctagccgagtagtgttgggtcgcgaaaggccttgtggtactgcctgatagggtgcttgcgagtgccccgggaggtctcgtagaccgtgcaacatgagcacgaatcctaaacctcaaagaaaaaccaaaagaaacaccaaccgccg

SEQ ID NO: 50 - DNA, 197 bp, Hepatitis C Virus; identical to SEQ ID NO: 48
  source 1..197: mol_type = other DNA

SEQ ID NO: 51 - DNA, 198 bp, Hepatitis C Virus
  source 1..198: mol_type = other DNA
ttggaacaaccccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacacttcctaaacctcaaagaataaccaaaagaaacaccaaccgtcg

SEQ ID NO: 52 - DNA, 200 bp, Hepatitis C Virus
  source 1..200: mol_type = other DNA
cattggatcaaacccgctcaatgcctggagatttgggcgtgcccccgcaagactgctagccgagtagcgttgggttgcgaaaggccttgtggtactgcctgatagggtgcttgcgagtgccccgggaggtctcgtagaccgtgcatcatgagcacacttccaaaaccccaaagaaaaaccaaaagaaacaccaaccgtcg

SEQ ID NO: 53 - DNA, 200 bp, Hepatitis C Virus
  source 1..200: mol_type = other DNA
cattggatcaaacccgctcaatgcctggagatttgggcgtgcccccgcaagactgctagccgagtagcgttgggttgcgaaaggccttgtggtactgcctgatagggtgcttgcgagtgccccgggaggtctcgtagaccgtgcaacatgagcacacttcctaaacctcaaagaaaaaccaaaagaaacaccaaccgtcg

SEQ ID NO: 54 - DNA, 198 bp, Hepatitis C Virus
  source 1..198: mol_type = other DNA
ttggatcaaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacacttccaaaaccccaaagaaaaaccaaaagaaacaccaaccgtcg

SEQ ID NO: 55 - DNA, 197 bp, Hepatitis C Virus
  source 1..197: mol_type = other DNA
ttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacacttccaaaaccccaaaaaagaaaccaaagaaacacaaaccgtcg

SEQ ID NO: 56 - DNA, 197 bp, Hepatitis C Virus
  source 1..197: mol_type = other DNA
ttggatyaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacacttcctaaacctcaaagaaaaaccaaaagaaacacaacccgccg

SEQ ID NO: 57 - DNA, 197 bp, Hepatitis C Virus
  source 1..197: mol_type = other DNA
ttggattaacccgctcagtgcctggagatttgggcgtgcccccgcgagaccgctagccgagtagtgttgggtcgcgaaaggccttgtggtactgcctgatagggtgcttgcgagtgccccgggaggtctcgtagaccgtgcatcatgagcacacttccaaaaccccaaagaaaaaccaaaagaaacaccaaccgccg

SEQ ID NO: 58 - DNA, 197 bp, Hepatitis C Virus
  source 1..197: mol_type = other DNA
ttggattaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatccaaaaccccaaagacaaaccaaaagaaacaccaaccgtcg

SEQ ID NO: 59 - DNA, 197 bp, Hepatitis C Virus
  source 1..197: mol_type = other DNA
ttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcatcatgagcacacttccaaaaccccaaagaaaaaccaaaagaaacactaaccgtcg

SEQ ID NO: 60 - DNA, 197 bp, Hepatitis C Virus
  source 1..197: mol_type = other DNA
ttggattaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaycatgagcacacttccaaaaccccaaagaaaaaccaaaagaaacactaaccgtcg

SEQ ID NO: 61 - DNA, 197 bp, Hepatitis C Virus
  source 1..197: mol_type = other DNA
ttggattaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacacttccaaaaccccaaagacaaaccaaaagaaacaccaaccgccg

SEQ ID NO: 62 - DNA, 197 bp, Hepatitis C Virus
  source 1..197: mol_type = other DNA
ttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacacttccaaaacccctaagaaaaaccaaaagaaacaccaaccgtct